(19) United States
(12) Patent Application Publication
(10) Pub. No.: US 2019/0231230 A1
(43) Pub. Date: Aug. 1, 2019

(54) CEREBRAL FUNCTION STATE EVALUATION DEVICE BASED ON BRAIN HEMOGLOBIN INFORMATION

(71) Applicant: SOOCHOW UNIVERSITY, Suzhou (CN)

(72) Inventors: Chunguang LI, Suzhou (CN); Lining SUN, Suzhou (CN)

(21) Appl. No.: 16/341,430

(22) PCT Filed: Feb. 26, 2018
(86) PCT No.: PCT/CN2018/077176

§ 371 Date: Apr. 11, 2019

(30) Foreign Application Priority Data
Jan. 30, 2018 (CN) 201810090704.0

(51) Int. Cl. A61B 5/11; G06N 20/10; A61B 5/1455; A61B 5/00; G06K 9/62

(52) U.S. Cl. CPC A61B 5/1124; G06N 20/10; A61B 2503/08; A61B 5/726; G06K 9/6229; A61B 5/14553

(57) ABSTRACT

The present invention relates to a cerebral function state evaluation device, which comprises a brain oxyhemoglobin concentration variation acquiring component, acquiring brain oxyhemoglobin concentrations of a stroke patient, who is in a phase of completing finger-nose and heel-knee-tibia tests under instruction, by applying near-infrared spectroscopic brain imaging technology; a brain functional network constructing component; a typical feature acquiring component; and an evaluation model establishing component. The cerebral function state evaluation device evaluates a patient's motor ability based on brain hemoglobin information. By using the proposed evaluation device, an evaluation result can be given only if a patient completes several required actions. The device is inventive and simple to operate, and subjective factors in the process of the scale scoring can be avoided.

### FIELD OF THE INVENTION

The present invention relates to evaluation of cerebral function state, and particularly to a cerebral function state evaluation device.

### BACKGROUND OF THE INVENTION

Cerebrovascular disease is one of the main diseases affecting the physical and mental health of middle-aged and elderly people. The most notorious disease among cerebrovascular diseases is cerebral apoplexy, also known as “stroke.” At present, the development trend of cerebral apoplexy in China is really serious, with more than 1.5 million new cases diagnosed every year. In addition, cerebral apoplexy has high disability rate. According to a latest report, 75% of these newly cases diagnosed lost their ability to work, which has a huge impact on both the patients and society. Therefore, to assist doctors in targeted training and treatment and help patients recover better, objective assessment on the recovery of patients' motor function has become a major and urgent task.

In order to assess the recovery of motor function of stroke patients, it's common to score the patients' recovery by using an assessment scale such as Fugl-Meyer, Berg Balance Scale, and Brunnstrom_6 stage assessment in clinical medicine, wherein the Fugl-Meyer assessment is the most common and reliable. However, these scoring methods, including Fugl-Meyer, still have some shortcomings. For example, the patient is required to actively cooperate with treatment, and the motion evaluation of the trunk portion is neglected. These require a large amount of time and effort of the medical staff, and the scores are given by the work staff with high subjectivity. Therefore, it is urgent to propose a scientific and objective simple method to assess the recovery of patients' motor function.

The brain activity of the patient can be objectively recorded by brain imaging technologies. At present, the most widely used brain imaging technologies are fMRI, EEG, and fNIRS. Although the brain imaging technologies such as fMRI and PET have high spatial resolution, exercise test with large motion scale of limbs are not supported, which has limitations on the evaluation of motor function. The EEG technology is highly advantageous in time resolution, but suffers from the problem of traceability, which is not conducive to the location of affected brain functional regions. The fNIRS technology has some advantages compared to other technologies such as supporting exercise test, not susceptible to the test environment, portable and flexible. Therefore, the use of advanced brain imaging technologies is important in scientifically and objectively assessing the recovery level of the patients' motor function.

By constructing a brain functional network with brain imaging signals, the recovery level of the patients can be scientifically analyzed. At present, there are various methods for analyzing brain signals, including monitoring the brain activity and state by analyzing the positive and negative activation of the brain regions and monitoring the brain region connectivity by calculating the brain functional connectivity. However, these methods cannot reflect the deeper inherent operating mechanism of the brain. The model constructed by a method for constructing brain functional network can greatly approximate the real state of brain activity, so that the damage and recovery degree of the brain can be effectively and scrupulously analyzed to evaluate the recovery of the patient's motor ability. Therefore, using topological theory to construct a functional network for the brain and analyze the brain activity is important in scientifically and objectively assessing the recovery level of motor function.

### SUMMARY OF THE INVENTION

In view of this, to solve the above technical problems, the present invention proposes an evaluation device for recovery level of motor function of stroke patients based on brain hemoglobin information, to evaluate stroke patients at different recovery levels and help to realize more modern intelligent medical aids for recovery.

A cerebral function state evaluation device comprises:

a brain oxyhemoglobin concentration variation acquiring component, acquiring brain oxyhemoglobin concentrations of a stroke patient who is in a phase of completing finger-nose and heel-knee-tibia tests under instruction by applying near-infrared spectroscopic brain imaging technology;

a brain functional network constructing component, evaluating functional connection of the brain by analyzing oxyhemoglobin concentrations acquired by the brain oxyhemoglobin concentration variation acquiring component, and constructing a brain functional network therewith;

a typical feature acquiring component, calculating network topology parameters of the brain functional network constructed by the brain functional network constructing component; these network topology parameters, combined with the wavelet coherence coefficients between brain regions, are considered as an original feature set; the original feature set is screened by filtering and cooperative wrapper-based feature selection methods, and final typical features are obtained; and

an evaluation model establishing component, fitting the final typical features acquired by the typical feature acquiring component and establishing an evaluation model of recovery level of the stroke patient by using a machine learning algorithm of a support vector regression machine.

The cerebral function state evaluation device evaluates a patient's motor ability based on brain hemoglobin information. By using the proposed evaluation device, an evaluation result can be given only if the patient completes several required actions. The device is inventive and simple to operate, and subjective factors in the process of the scale scoring can be avoided.

In another embodiment, in the “completing finger-nose and heel-knee-tibia tests under instruction,” the upper limbs perform the finger-nose action task, and the lower limbs perform the heel-knee-tibia task, and the upper and lower limbs on both the healthy and affected side respectively perform respective task for 4 times, where the rest time between every two tasks is 30 seconds.

In another embodiment, in the “a brain functional network constructing component, evaluating functional connection of the brain by analyzing oxyhemoglobin concentrations acquired by the brain oxyhemoglobin concentration variation acquiring component, and constructing a brain functional network therewith,” when evaluating the functional connection of the brain, wavelet coherence analysis method is used to calculate the coherence of each brain functional region, and the coherence coefficients are used to evaluate the functional connection of the brain.

In another embodiment, in the “a brain functional network constructing component, evaluating functional connection of the brain by analyzing oxyhemoglobin concentrations acquired by the brain oxyhemoglobin concentration variation acquiring component, and constructing a brain functional network therewith,” when the brain functional network is constructed, network parameters of the functional network are calculated, including an average node degree, a network density, and a clustering coefficient.

In another embodiment, in the “a typical feature acquiring component, calculating network topology parameters of the brain functional network constructed by the brain functional network constructing component; these network topology parameters, combined with the wavelet coherence coefficients between brain regions, are considered as an original feature set; the original feature set is screened by filtering and cooperative wrapper-based feature selection methods, and final typical features are obtained,” network parameters of different brain regions are compared respectively, and digital feature values of the network parameters are calculated, and the digital feature values include a covariance, a mean square error and a mean; and a corresponding mean, variance and coefficient of variation are calculated based on the coherence coefficient between brain regions calculated “in the ‘a brain functional network constructing component, evaluating functional connection of the brain by analyzing oxyhemoglobin concentrations acquired by the brain oxyhemoglobin concentration variation acquiring component, and constructing a brain functional network therewith,’ when evaluating the functional connection of the brain, wavelet coherence analysis method is used to calculate the coherence of each brain functional region, and the coherence coefficients are used to evaluate the functional connection of the brain,” all the above values corresponding to network parameters and coherence coefficients are combined as the original feature set.

In another embodiment, in the “a typical feature acquiring component, calculating network topology parameters of the brain functional network constructed by the brain functional network constructing component; these network topology parameters, combined with the wavelet coherence coefficients between brain regions, are considered as an original feature set; the original feature set is screened by filtering and cooperative wrapper-based feature selection methods, and final typical features are obtained,” when the original feature set is screened by using a feature selection method, the feature set is firstly preliminarily screened by a filter-based feature selection method; and then typical features are further selected from those preliminarily screened as the final typical features, by a wrapper-based feature selection method.

In another embodiment, the filter-based feature selection method is a correlation coefficient method.

In another embodiment, the wrapper-based feature selection method is a genetic algorithm.

### BRIEF DESCRIPTION OF THE DRAWINGS

FIG. 1 is a schematic structural diagram of a cerebral function state evaluation device according to an embodiment of the present application.

FIG. 2 is a flow chart of a genetic algorithm in a cerebral function state evaluation device according to an embodiment of the present application.

### DETAILED DESCRIPTION OF THE EMBODIMENTS

To make the objects, technical solutions, and advantages of the present invention clearer, the present invention is described in further detail with reference to accompanying drawings and examples. It should be understood that the specific examples described herein are merely provided for illustrating, instead of limiting the present invention.

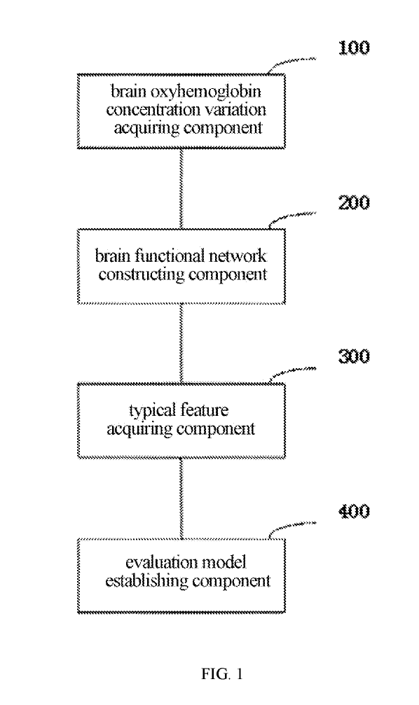

Referring to FIG. 1, a cerebral function state evaluation device comprises:

a brain oxyhemoglobin concentration variation acquiring component 100, acquiring brain oxyhemoglobin concentrations of a stroke patient who is in a phase of completing finger-nose and heel-knee-tibia tests under instruction by applying near-infrared spectroscopic brain imaging technology;

a brain functional network constructing component 200, evaluating functional connection of the brain by analyzing oxyhemoglobin concentrations acquired by the brain oxyhemoglobin concentration variation acquiring component, and constructing a brain functional network therewith;

a typical feature acquiring component 300, calculating network topology parameters of the brain functional network constructed by the brain functional network constructing component; these network topology parameters, combined with the wavelet coherence coefficients between brain regions, are considered as an original feature set; the original feature set is screened by filtering and cooperative wrapper-based feature selection methods, and the final typical features are obtained; and an evaluation model establishing component 400, fitting the final typical features acquired by the typical feature acquiring component and establishing an evaluation model of recovery level of the stroke patient by using a machine learning algorithm of a support vector regression machine.

When a machine learning algorithm is used, the machine learning algorithm of a support vector regression machine is used to learn and fit the features obtained by the typical feature acquiring component, and establish an evaluation model.

Specifically, for a given training sample D={(x₁,y₁), (x₂,y₂), . . . , (xₘ,yₘ)}, the support vector regression machine is intended to obtain a formula below to fit the sample:

f(x)=ω^{T}x+b

The above problem can be converted into the following problem:

$${min}_{ω , b , {ξ}_{i} , {\hat{ξ}}_{i}} \frac{1}{2} {‖ ω ‖}^{2} + C {\sum }_{i = 1}^{m} ( {ξ}_{i} + {\hat{ξ}}_{i} )$$

$$s . t . \{ \begin{matrix}f ( {x}_{i} ) - {y}_{i} \leq ϵ + {ξ}_{i} \\ {y}_{i} - f ( {x}_{i} ) \leq ϵ + {ξ}_{i} \\ {ξ}_{i} \geq 0 , {\hat{ξ}}_{i} \geq 0 , i = 1 , 2 , … , m\end{matrix}$$

The Lagrangian multiplier is introduced, and the dual problem obtained is:

$${max}_{α , \hat{α}} {\sum }_{i = 1}^{m} {y}_{i} ( {\hat{α}}_{i} - {α}_{i} ) - ϵ ( {\hat{α}}_{i} + {α}_{i} ) - \frac{1}{2} {\sum }_{i = 1}^{m} {\sum }_{j = 1}^{m} ( {\hat{α}}_{i} - {α}_{i} ) ( {\hat{α}}_{j} - {α}_{j} ) K ( {x}_{1} {x}_{j} )$$

$$s . t . \{ \begin{matrix}{\sum }_{i = 1}^{m} ( {\hat{α}}_{i} - {α}_{i} ) \\ 0 \leq {α}_{i} {\hat{, α}}_{i} \leq C\end{matrix}$$

The Lagrange multiplier is solved to obtain the offset:

$$b = {y}_{i} + ɛ - {\sum }_{i = 1}^{m} ( {\hat{α}}_{i} - {α}_{i} ) K ( {x}_{i} , {x}_{j} )$$

Then, the final fitting curve is:

$$f ( x ) = {\sum }_{i = 1}^{m} ( {\hat{α}}_{i} - {α}_{i} ) K ( {x}_{i} , {x}_{j} ) + b$$

It is to be understood that the components such as the brain oxyhemoglobin concentration variation acquiring component, the brain functional network constructing component, the typical feature acquiring component, the evaluation model establishing component, and the like can be implemented with hardware. Those skilled in the art should understand how to implement the above components through hardware (for example, discrete hardware elements, integrated circuits, digital circuits based on gate devices, analog circuit components, programmable hardware devices (such as microcontrollers, and FPGAs, etc.) and circuit systems composed of any combination of the above).

The cerebral function state evaluation device evaluates a patient's motor ability based on brain hemoglobin information. By using the proposed evaluation device, an evaluation result can be given only if the patient completes several required actions. The device is inventive and simple to operate, and subjective factors in the process of the scale scoring can be avoided.

In another embodiment, in the “completing finger-nose and heel-knee-tibia tests under instruction,” the upper limbs perform the finger-nose task, the lower limbs perform the heel-knee-tibia task, and the upper and lower limbs on both the healthy and affected side respectively perform the respective task for 4 times, where the rest time between two tests is 30 seconds.

In another embodiment, in the “a brain functional network constructing component, evaluating functional connection of the brain by analyzing oxyhemoglobin concentrations acquired by the brain oxyhemoglobin concentration variation acquiring component, and constructing a brain functional network therewith,” when evaluating the functional connection of the brain, wavelet coherence analysis method is used to calculate the coherence of each brain functional region, and the coherence coefficients are used to evaluate the functional connection of the brain.

Specifically, in the pre-processing, a method of mathematical morphology filtering is used to perform baseline correction on an original signal, and then a moving average smoothing method is used to remove high-frequency components in the signal:

Input sequence f(n) and structural element k(m) are defined.

Erosion operation is defined as:

(f⊖k)(n)=min_{m=0, . . . ,M-1}{f(n+m)−k(m)}n=0,1, . . . ,N−M

Dilation operation is defined as:

(f⊕k)(n)=max_{m=0, . . . ,M-1}{f(n−m)+k(m)}n=0,1, . . . ,N−M

Morphological opening operation is defined as:

(f∘k)(n)=[(f⊖k)⊕k](n)

Morphological closing operation is defined as:

(f·k)(n)=[(f⊕k)⊖k](n)

Signal after baseline correction f_{correction}:

f_{correction}=f₀(f₀∘k+f₀·k)/2

in which f₀ is the original signal.

Then the signal f_{correction} is smoothed to obtain a preprocessed signal f_{preprocess}:

f_{preprocess}=smooth(f_{correction})

in which smooth(⋅) is a moving averaging operator.

When evaluating the functional connection of the brain, wavelet coherence analysis method is used to calculate the coherence of each brain functional region at a center frequency of 0.04 Hz, and the functional connection of the brain is evaluated with the coherence coefficients.

Morlet wavelet is defined as:

Ψₘₒᵣₗₑₜ(η)=π^{-1/4}e^{-iω}^{₀}^{η}e^{-η}^{²}^{/2}

Continuous wavelet transform is defined as:

$${W}_{n} ( s ) = {\sum }_{n = 0}^{N - 1} {x}_{n} Ψ ⋆ [ \frac{( {n}^{′} - n ) {δ}_{t}}{s} ]$$

Discrete Fourier transform is performed on xₙ, and according to the convolution theory, it is obtained that:

$${W}_{n} ( s ) = {\sum }_{k = 0}^{N - 1} {x}_{k} Ψ ⋆ ( s {ω}_{k} ) {e}^{i {ω}_{k} n {δ}_{t}}$$

in which the angular frequency is defined as:

$${ω}_{k} = \{ \begin{matrix}\frac{2 π k}{N {δ}_{t}} : k \leq \frac{N}{2} \\ - \frac{2 π k}{N {δ}_{t}} : k > \frac{N}{2} :\end{matrix}$$

The smoothing operation over time is defined as Sₜᵢₘₑ:

$${\overline{W}}_{n}^{2} ( s ) = \frac{1}{{n}_{a}} {\sum }_{n = {n}_{1}}^{{n}_{2}} {\vert {W}_{n} ( s ) \vert}^{2}$$

The smoothing operation over scale is defined as S_{scale}: scale:

$${\overline{W}}_{n}^{2} = \frac{{δ}_{j} {δ}_{t}}{{C}_{δ}} {\sum }_{j = {j}_{1}}^{{j}_{2}} \frac{{\vert {W}_{n} ( {s}_{j} ) \vert}^{2}}{{s}_{j}}$$

The smoother is defined as:

S(W)=S_{scale}(Sₜᵢₘₑ(Wₙ(s)))

The cross spectrum is defined as:

Wₙ^{XY}(s)=|Wₙ^{X}(s)Wₙ^{Y*}(s)|

Wavelet coherence coefficient:

$${R}_{n}^{2} ( s ) = \frac{{\vert S ( {s}^{- 1} {W}_{n}^{XY} ( s ) ) \vert}^{2}}{S ( {s}^{- 1} {\vert {W}_{n}^{X} ( s ) \vert}^{2} ) S ( {s}^{- 1} {\vert {W}_{n}^{Y} ( s ) \vert}^{2} )}$$

In another embodiment, in the “a brain functional network constructing component, evaluating functional connection of the brain by analyzing oxyhemoglobin concentrations that acquired by the brain oxyhemoglobin concentration variation acquiring component, and constructing a brain functional network therewith,” when the brain functional network is constructed, network parameters of the functional network are calculated, including an average node degree, a network density, and a clustering coefficient.

Specifically, when the brain functional network is constructed, a threshold is set according to the strength of functional connection of the brain, an adjacency matrix is obtained based on the threshold, and network parameters such as an average node degree, a network density, and a clustering coefficient of the functional network are calculated based on the value of the adjacency matrix.

The adjacency matrix is calculated:

$${M}_{adj} ( i , j ) = \{ \begin{matrix}1 & R ( i , j ) \geq T \\ 0 & R ( i , j ) < T\end{matrix}$$

in which R(i,j) is a wavelet coherence value of a channel i and a channel j, which are calculated with the following equation

$${R}_{n}^{2} ( s ) = \frac{{\vert S ( {s}^{- 1} {W}_{n}^{XY} ( s ) ) \vert}^{2}}{S ( {s}^{- 1} {\vert {W}_{n}^{X} ( s ) \vert}^{2} ) S ( {s}^{- 1} {\vert {W}_{n}^{Y} ( s ) \vert}^{2} )}$$

and T is a set threshold.

The brain functional network is constructed according to the adjacency matrix, and then the following three brain functional network parameters are calculated:

N is defined as the number of nodes in the network, and when T is given, they are calculated as follows.

Average node degree:

$$K = {Φ}_{degree} ( {M}_{adj} ) = \frac{1}{N} {\sum }_{i = 1}^{N} {K}_{i}$$

Kᵢ represents a degree value at each node i.

$${K}_{i} = {\sum }_{j = 1 , j \neq i}^{N} {M}_{adj} ( i , j )$$

Network density:

$$D = {Φ}_{density} ( {M}_{adj} ) = \frac{{\sum }_{i = 1 , i \neq j}^{N} {\sum }_{j = 1 , j \neq i}^{N} {M}_{adj} ( i , j )}{2 N ( N - 1 )}$$

Clustering coefficient of a node i:

$${C}_{i} = \frac{2 {e}_{i}}{{K}_{i} ( {K}_{i} - 1 )}$$

eᵢ represents the number of nodes adjacent to the node i

Clustering coefficient of the network:

$$C = {Φ}_{clustercoff} ( {M}_{adj} ) = \frac{1}{N} {\sum }_{i = 1}^{N} {C}_{i}$$

In another embodiment, in the “a typical feature acquiring component, calculating network topology parameters of the brain functional network constructed by the brain functional network constructing component; these network topology parameters, combined with the wavelet coherence coefficients between brain regions, are considered as the original feature set; the original feature set is screened by filtering and cooperative wrapper-based feature selection methods, and the final typical features are obtained,” network parameters of different brain regions are compared respectively, and digital feature values of the network parameters are calculated, and the digital feature values include a covariance, a mean square error and a mean; and a corresponding mean, variance and coefficient of variation are calculated based on the coherence coefficient between brain regions calculated “in the ‘a brain functional network constructing component, evaluating functional connection of the brain by analyzing oxyhemoglobin concentrations acquired by the brain oxyhemoglobin concentration variation acquiring component, and constructing a brain functional network therewith,’ when evaluating the functional connection of the brain, wavelet coherence analysis method is used to calculate the coherence of each brain functional region, and the coherence coefficients are used to evaluate the functional connection of the brain,” all the above values corresponding to network parameters and coherence coefficients are combined as the original feature set.

When the digital features between the parameter variation curves and the wavelet coherence values between brain regions are calculated, the network parameter variation curves of different brain regions and the network parameter variation curves of the healthy side and the affected side during the task phase are compared, and a covariance, a mean square error, a volatility, a mean, and other digital feature values between variation curves are calculated and used as a feature set. In addition, the mean, variance and coefficient of variation of the coherence values between the brain regions are also added to the feature set.

The following parameters between curves are calculated:

Covariance:

$$Cov ( X , Y ) = \frac{1}{N - 1} {\sum }_{T = {t}_{1}}^{{t}_{2}} ( {Φ}_{X} ( T ) - \overline{{Φ}_{X}} ) ( {Φ}_{Y} ( T ) - \overline{{Φ}_{Y}} )$$

Root mean square error:

$$RMS E ( X , Y ) = \sqrt{\frac{1}{N} {\sum }_{T = {t}_{1}}^{{t}_{2}} {Φ}_{X} ( T ) - {Φ}_{Y} ( T )}$$

Mean:

$$E ( X ) = \frac{1}{N} {\sum }_{T = {t}_{1}}^{{t}_{2}} {Φ}_{X} ( T )$$

Volatility:

$$Flu ( X ) = \frac{1}{N} {\sum }_{T = {t}_{1}}^{{t}_{2}} \vert d ( {Φ}_{X} ( T ) ) \vert$$

In another embodiment, in the “a typical feature acquiring component, calculating network topology parameters of the brain functional network constructed by the brain functional network constructing component; these network topology parameters, combined with the wavelet coherence coefficients between brain regions, are considered as the original feature set; the original feature set is screened by filtering and cooperative wrapper-based feature selection methods, and the final typical features are obtained,” when the original feature set is screened by using a feature selection method, the feature set is firstly preliminarily screened by a filter-based feature selection method; and then typical features are further selected from those preliminarily screened as the final typical features, by a wrapper-based feature selection method.

In another embodiment, the filter-based feature selection method is a correlation coefficient method.

Specifically, a sample set D={(x₁,y₁), (x₂,y₂), . . . , (xₘ,yₘ)} is given, in which xᵢ is a feature set and yᵢ is a true value.

A Pearson correlation coefficient operator Peason(⋅) is defined, and the square of the Pearson correlation coefficient of each column of features in the feature set with the true values r² is calculated as follows:

r²(i)=Peason(xⁱ,y)²

r² is sorted, and the features corresponding to the 25 highest r² are used as preliminary features.

In another embodiment, the wrapper-based feature selection method is a genetic algorithm.

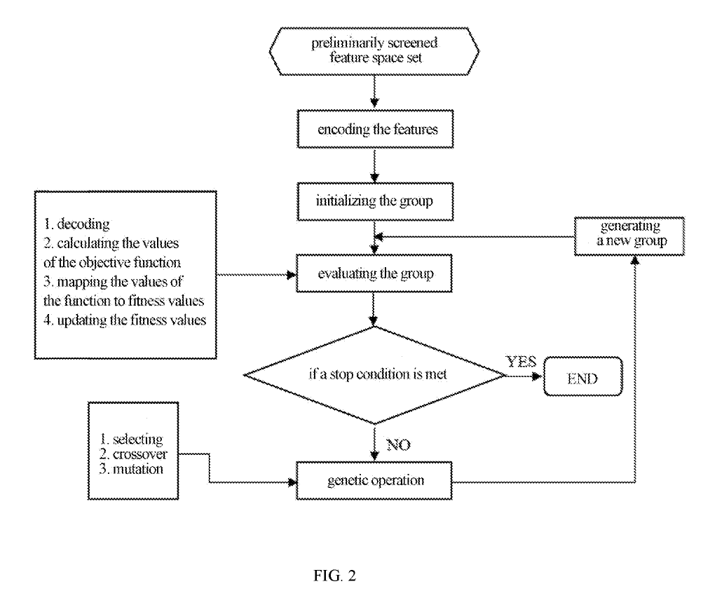

For specific steps of the genetic algorithm, reference may be made to FIG. 2.

In the present invention, wavelet coherence analysis method is used, and the correlation between nodes in a central frequency band can be analyzed, which is beneficial to monitoring the correlation between brain regions in each neural activity frequency band and physiologically active frequency band.

In the present invention, complex network analysis method is used, and the cooperation between various brain regions is analyzed from the perspective of the data transmission capability and the work efficiency between the nodes, which is beneficial to finding the parameter index reflecting the working state of the brain.

In the present invention, the algorithm of support vector regression machine is used, and an optimal regression model can be established according to the information of the feature parameters, thereby improving the accuracy of determining the state of the brain.

In the present invention, the patient's motor ability is evaluated based on the brain information. Based on the proposed evaluation device, an evaluation result can be given only if the patient completes several required actions. The device is inventive and simple to operate, and subjective factors during the evaluation can be avoided.

In the present invention, the near-infrared spectroscopic brain imaging technology is used for test, which is simple in operation. It has low requirements on the external environment, and low susceptibility to environmental noise. The subjects will not be negatively affected. Throughout the test, the patient completes the finger-nose and heel-knee-tibia tests in the natural environment, and the resulting analysis results are more reliable to assess the patient's recovery level.

The technical features of the above-described embodiments may be used in combination. For the sake of brevity, not all possible combinations of the technical features in the above embodiments are described. However, where no contradiction exists, all the combinations of these technical features are contemplated in the scope of the present invention.

The above-described embodiments are merely illustrative of several implementations of the present invention, and the description is specific and particular, but is not to be construed as limiting the scope of the present invention. It should be pointed out that for those of ordinary skill in the art, several variations and improvements can be made without departing from the concept of the present invention, all of which fall within the protection scope of the present invention. Therefore, the protection scope of the present invention is defined by the appended claims.

What is claimed is:

1. A cerebral function state evaluation device, comprising:
a brain oxyhemoglobin concentration variation acquiring component, acquiring brain oxyhemoglobin concentrations of a stroke patient, who is in a phase of completing finger-nose and heel-knee-tibia tests under instruction by applying near-infrared spectroscopic brain imaging technology;
a brain functional network constructing component, evaluating functional connection of the brain by analyzing oxyhemoglobin concentrations that acquired by the brain oxyhemoglobin concentration variation acquiring component, and constructing a brain functional network therewith;
a typical feature acquiring component, calculating network topology parameters of the brain functional network constructed by the brain functional network constructing component; these network topology parameters, combined with the wavelet coherence coefficients between brain regions, are considered as an original feature set; the original feature set is screened by filtering and cooperative wrapper-based feature selection methods, and the final typical features are obtained, and
an evaluation model establishing component, fitting the final typical features acquired by the typical feature acquiring component, and establishing an evaluation model of recovery level of the stroke patient by using a machine learning algorithm of a support vector regression machine.

2. The cerebral function state evaluation device according to claim 1, wherein in the “completing finger-nose and heel-knee-tibia tests under instruction,” the upper limbs perform the finger-nose action task, and the lower limbs perform the heel-knee-tibia task, and the upper and lower limbs on both the healthy and affected side respectively perform respective task for 4 times, where the rest time between every two tasks is 30 seconds.

3. The cerebral function state evaluation device according to claim 1, wherein in the “a brain functional network constructing component, evaluating functional connection of the brain by analyzing oxyhemoglobin concentrations that acquired by the brain oxyhemoglobin concentration variation acquiring component, and constructing a brain functional network therewith,” when evaluating the functional connection of the brain, wavelet coherence analysis method is used to calculate the coherence of each brain functional region, and the coherence coefficients are used to evaluate the functional connection of the brain.

4. The cerebral function state evaluation device according to claim 1, wherein in the “a brain functional network constructing component, evaluating functional connection of the brain by analyzing oxyhemoglobin concentrations that acquired by the brain oxyhemoglobin concentration variation acquiring component, and constructing a brain functional network therewith,” when the brain functional network is constructed, network parameters of the functional network are calculated, including an average node degree, a network density, and a clustering coefficient.

5. The cerebral function state evaluation device according to claim 3, wherein in the “a typical feature acquiring component, calculating network topology parameters of the brain functional network constructed by the brain functional network constructing component; these network topology parameters, combined with the wavelet coherence coefficients between brain regions, are considered as an original feature set; the original feature set is screened by filtering and cooperative wrapper-based feature selection methods, and the final typical features are obtained,” network parameters of different brain regions are compared respectively, and digital feature values of the network parameters are calculated, and the digital feature values include a covariance, a mean square error and a mean; and a corresponding mean, variance and coefficient of variation are calculated based on the coherence coefficient between brain regions calculated “in the ‘a brain functional network constructing component, evaluating functional connection of the brain by analyzing oxyhemoglobin concentrations that acquired by the brain oxyhemoglobin concentration variation acquiring component, and constructing a brain functional network therewith,’ when evaluating the functional connection of the brain, wavelet coherence analysis method is used to calculate the coherence of each brain functional region, and the coherence coefficients are used to evaluate the functional connection of the brain,” all the above values corresponding to network parameters and coherence coefficients are combined as the original feature set.

6. The cerebral function state evaluation device according to claim 1, wherein in the “a typical feature acquiring component, calculating network topology parameters of the brain functional network constructed by the brain functional network constructing component; these network topology parameters, combined with the wavelet coherence coefficients between brain regions, are considered as an original feature set; the original feature set is screened by filtering and cooperative wrapper-based feature selection methods, and the final typical features are obtained,” when the original feature set is screened by using a feature selection method, the feature set is firstly preliminarily screened by a filter-based feature selection method; and then typical features are further selected from those preliminarily screened as the final typical features, by a wrapper-based feature selection method.

7. The cerebral function state evaluation device according to claim 6, wherein the filter-based feature selection method is a correlation coefficient method.

8. The cerebral function state evaluation device according to claim 6, wherein the wrapper-based feature selection method is a genetic algorithm.
